(19) United States
(12) Patent Application Publication
(10) Pub. No.: US 2005/0014165 A1
(43) Pub. Date: Jan. 20, 2005

(54) Biomarker panel for colorectal cancer

(75) Inventors: Nancy M. Lee, San Francisco, CA (US); Ling C. Chen, Fremont, CA (US)

Correspondence Address: COOLEY GODWARD, LLP, 3000 EL CAMINO REAL, 5 PALO ALTO SQUARE, PALO ALTO, CA, 94306, US

(73) Assignee: California Pacific Medical Center, San Francisco, CA (US)

(21) Appl. No.: 10/690,880

(22) Filed: Oct. 22, 2003

Related U.S. Application Data
(60) Provisional application No. 60/488,660, filed on Jul. 18, 2003.

(51) Int. Cl. C12Q 1/68

(52) U.S. Cl. 435/6

(57) ABSTRACT

A panel of biomarkers has been identified for analysis of colorectal cancer. The panel, originally identified using a mouse colon cancer model, has been used to assess changes in human tissue from surgical and biopsy samples against a normal human control panel of biomarkers. The panel may be used for providing a cost effective, rapid, noninvasive procedure for risk assessment, early diagnosis, establishing prognosis, monitoring patient treatment, detecting relapse, and for the discovery of therapeutic intervention of colorectal cancer.

### REFERENCE TO RELATED APPLICATIONS

This application claims priority to provisional application Ser. No. 60/488660, entitled, “Molecular Marker Panel for Determination of Colorectal Cancer”, which was filed on Jul. 18, 2003, and incorporated herein by reference.

### BACKGROUND

The field of art of this disclosure concerns biomarkers for colorectal cancer (CRC). These biomarkers are useful for risk assessment, early detection, establishing prognosis, evaluation of intervention, recurrence of CRC, and discovery of therapeutic intervention, and methods of use thereof.

In the field of medicine, clinical procedures providing for the risk assessment and early detection of CRC have been long sought. Currently, CRC is the second leading cause of cancer-related deaths in the Western world. One picture that has clearly emerged through decades of research into CRC is that early detection is critical to enhanced survival rates.

The currently accepted methods for CRC screening include the fecal occult blood test (FOBT), x-ray using double contrast between barium enema and air (DCBE), sigmoidoscopy, and colonoscopy. Sigmoidoscopy is an invasive procedure that visually examines the lower third of the colon using a lighted, flexible endoscope, while a related method, colonoscopy, is a procedure that examines the entire colon. In both cases, biopsy samples can be taken during the procedure.

Concerning the accepted methods for screening, none clearly possess what is desired in a screening examination for CRC. While FOBT is rapid, it is a very general, and therefore a very non-specific screening method for CRC. Though DCBE has proven useful in specifically imaging abnormalities in the colon, the drawbacks of the DCBE method include: 1.) Patient discomfort in preparation of and during the examination, creating reluctance for compliance of DCBE as a screening method. 2.) Exposure of a patient to x-ray radiation, limiting DCBE in terms of frequency of use as a screening method. 3.) Research indicating that DCBE is more effective in detecting larger growths, which contraindicates its use for early detection. 4.) Biopsy samples cannot be taken during the procedure. 5.) Due to the cost involved, not all insurance providers pay for DCBE screening exams. Though sigmoidoscopy has gained favor from many physicians, the drawbacks of this method include: 1.) Patient discomfort in preparation of and during the examination, creating reluctance for compliance of sigmoidoscopy as a screening method. 2.) Due to the cost involved, not all insurance providers pay for sigmoidoscopy screening exams. 3.) Since only the lower third of the colon is inspected, there is a suggestion by studies that many significant lesions are in the proximal end of the colon, rendering sigmoidoscopy inadequate. Though colonoscopy addresses the issue of complete inspection of the colon, the drawbacks of colonoscopy as a screening method include: 1.) Creating even more patient discomfort than sigmoidoscopy, therefore generally requiring sedation, and thereby exacerbating the issue with patient compliance. 2.) Due to the cost involved, not all insurance providers pay for colonoscopy screening exams. 3.) There are risks of colonoscopy that include bleeding, and puncture of the lining of the colon.

Emerging spectroscopic technologies, such as magnetic resonance imaging and tomographic imaging each have drawbacks that are drawn from the list of drawbacks for the currently accepted screening methodologies.

Accordingly, there is a need in the art for approaches that have value in early detection and treatment of CRC that are cost effective, rapid, and minimally or noninvasive. Additional utility would be realized from an approach that would also serve as the basis for establishing prognosis, monitoring patient treatment, and detecting relapse, as well as the discovery of therapeutic intervention of CRC.

### BRIEF DESCRIPTION OF FIGURES

FIG. 1 is a summary of the sequence listings.

FIGS. 2A-2C show data that illustrate a panel of biomarkers for samples taken from adenomous polyps, and suspect tissues vs. normal controls. FIGS. 2A-2B are tables that compare the results of model studies done in mouse (2A) for a selection of members of the set of 22 biomarkers listed in the sequence listings with the comparable selection in of biomarkers for human subjects (2B). FIG 2C shows the multivariate analysis for 9 markers for 78 biopsies taken from 12 normal patients and 63 biopsies taken from 6 patients with CRC.

FIGS. 3B-3C show expression levels for representative biomarkers, IL-8 (3A), CXCR-2 (3B), and COX-2 (3C) for a series of samples taken from a human subject comparing a histologically identified cancerous lesion, a polyp, and an adjacent non-cancerous tissue vs. a normal control.

FIGS. 4A-4C show the results of multiple analysis across a 53 cm distance of a colon for a patient with CRC: 4A shows expression levels for IL-8; 4B shows expression levels for COX-2; and 4C shows expression levels for CXCR-2.

### DETAILED DESCRIPTION

Still another sought after approach apart from currently accepted methods for screening for CRC, has been the search for biomarkers that have value in detection and treatment of CRC. For more than four decades, since the discovery of alpha-fetoprotein (AFP) and carcinogenic embryonic antigen (CEA), the search for biomarkers for cancer detection and treatment in general has been in a state of evolution. Biomarkers for cancer have five potential uses in the management of patient care. Ideally, they would be used for risk assessment, for early diagnosis, for establishing prognosis, for monitoring treatment, and for detecting relapse. Additionally, such markers could play a valuable role in developing therapeutic interventions.

It is further advantageous for the sampling methods used in conjunction with biomarker analysis to be minimally invasive or non-invasive. Examples of such sampling methods include serum, stool, swabs, and the like. Non-invasive and minimally invasive methods increase patient compliance, and generally reduce cost.

Clinically, the two criteria that are important for assessing the effectiveness of biomarkers are selectivity and sensitivity. Selectivity of a biomarker defined clinically refers to percentage of patients correctly diagnosed. Sensitivity of a biomarker in a clinical context is defined as the probability that the disease is detected at a curable stage. Ideally, biomarkers would have 100% clinical selectivity and 100% clinical sensitivity. To date, no single biomarker has been identified that has an acceptably high degree of selectivity and sensitivity required to be effective in for the broad range of needs in patient care management. However, from the clinical perspective, single serum biomarkers, such as AFP and CEA have proven to provide value in some aspects of patient care management.

For example, elevated serum levels of CEA were first discovered in 1965 in patients with adenocarcinoma of the colon. Elevated levels can be found in a variety of benign and malignant conditions other than colon cancer. Additionally, the production of CEA by early localized tumors of the colon is in the normal range. Therefore CEA lacks both the sensitivity and selectivity required to be of value for risk assessment or early diagnosis. Further, elevated levels of CEA correlate poorly with colon tumor differentiation and stage, rendering CEA as a biomarker for prognosis of colon cancer of limited value. The two areas for which CEA has proven helpful clinically in managing patient care are in evaluating the effectiveness of treatment, and for detecting relapse. Illustrative of this, numerous studies have found that there is high correlation between elevated serum levels of CEA preceding clinical detection of recurrence of colon cancer. This has proven to be of value in managing the care of high-risk patents with second-look surgical procedures based on rising levels of CEA.

Currently, investigations across numerous areas of oncology research, including CRC, ovarian, breast, and head and neck, are finding increased sensitivity and selectivity in panels of markers. It is now generally held that many mutations must take place before normal cell processes are altered, resulting in a disease, such as cancer. Still, given the complexity of biological systems, discovery of panels useful in providing value in patient care management for CRC is in the nascent stage.

To date, a greater understanding of the biology of CRC has been gained through the research on adenomous polyposis coli (APC), p53, and Ki-ras genes, as well as the corresponding proteins, and related pathways involved regulation thereof. However, there is a distinct difference between research on a specific a gene, its expression, protein product, and regulation, and understanding what genes are critical to include in a panel used to for the analysis of CRC that is useful in the management of patient care for the disease. To date, panels that have been suggested for CRC are comprised of specific point mutations of the APC, p53, and Ki-ras, as well as BAT-26, which is a gene that is a microstatelite instability marker.

What is disclosed herein is based on studies conducted in mouse multiple intestinal neoplasia (MIN) model, in which expressions levels of genes were screened in adenomous polyps. In the mouse MIN subjects, a chemically induced mutation of the APC gene is effected. The normal control is defined by littermates for which there was no aberration of the APC gene, and are therefore designated wildtype. From studies based on the mouse MIN model, candidate genes were selected for studying human subjects. From these human subject studies, a panel of biomarkers is disclosed herein. Further, what is disclosed are methods for measuring gene and protein expression levels based on the panel. Additionally, another aspect of what is disclosed are kits which provide the reagents and instructions for measuring gene and protein expression levels based on the panel. The panel, methods and kits are useful in the management of patient care for CRC. Additionally, the panel, methods and kits are believed useful as the basis for discovery of therapeutic interventions for CRC.

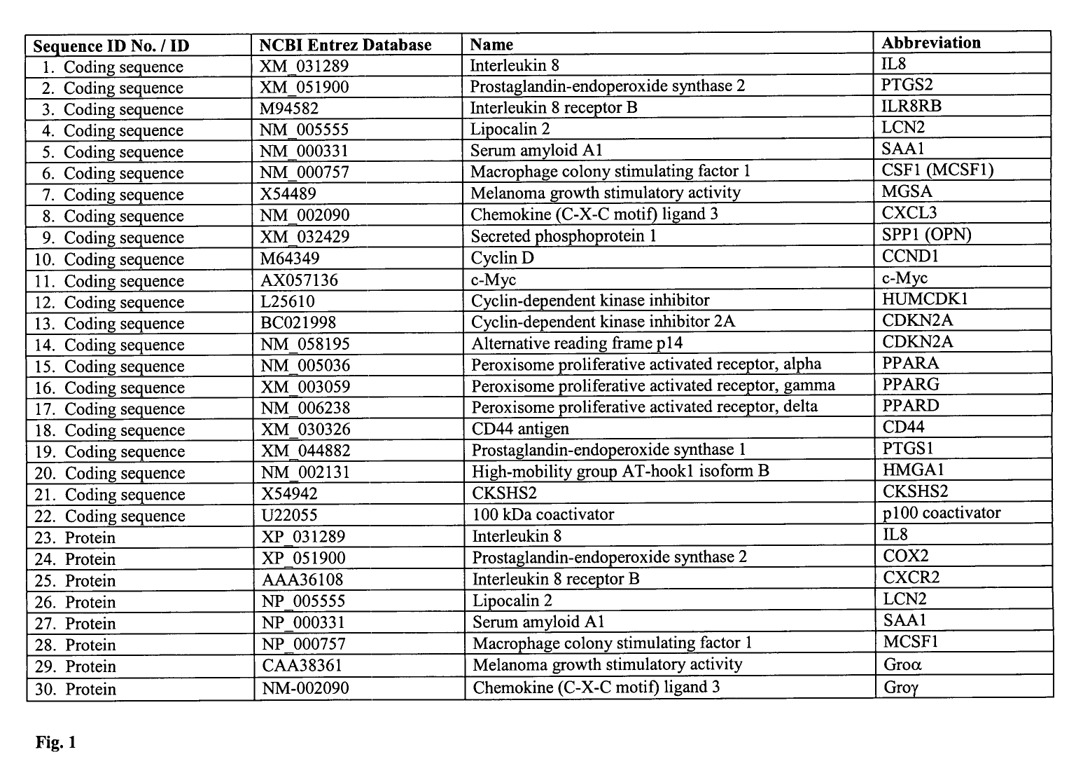

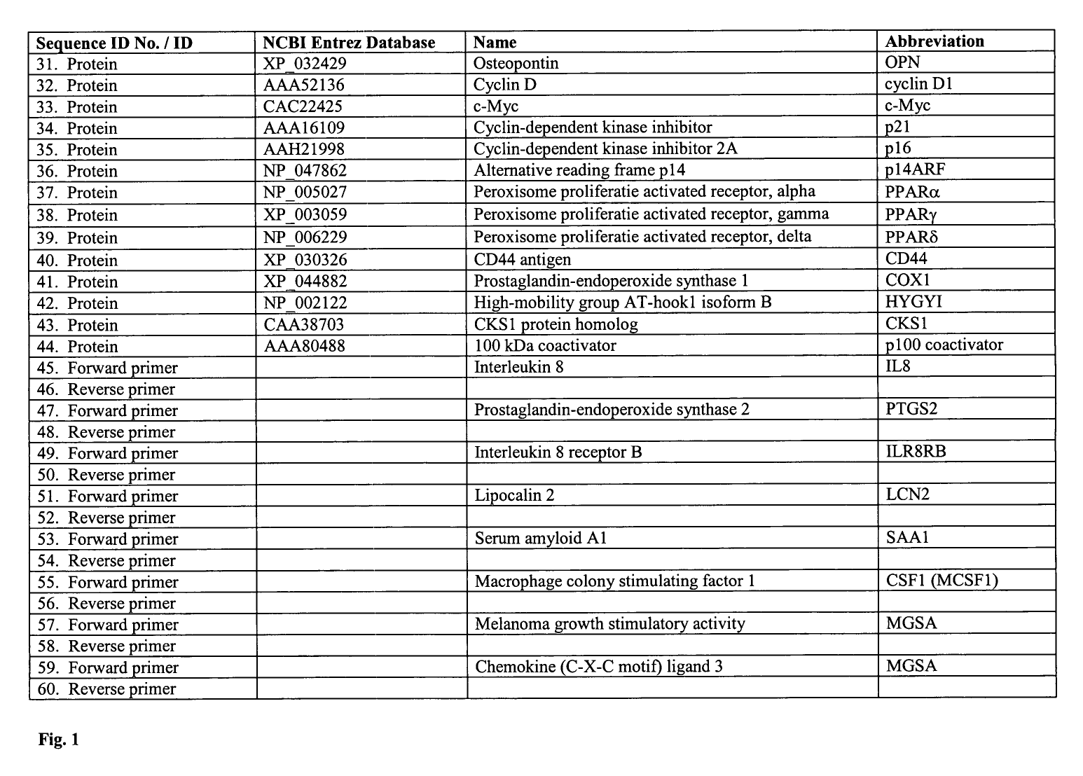

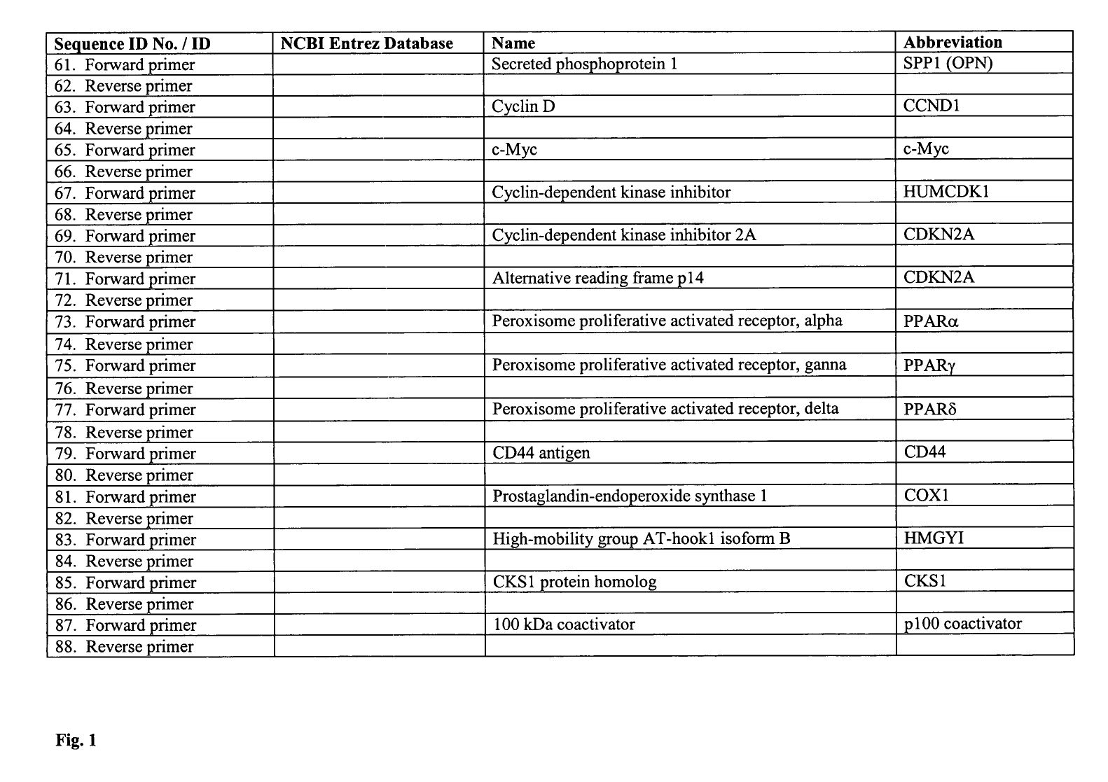

FIG. 1 is a table that gives an overview of the sequence listing for the disclosed biomarkers. The combination of biomarkers disclosed forms the basis for monitoring CRC with enhanced selectivity and sensitivity, and therefore providing enhanced management of patient care for CRC. It is to be understood that fragments and variants of the biomarkers described in the sequence listings are also useful biomarkers in a panel used for the analysis of CRC. What is meant by fragment is any incomplete or isolated portion of a polynucleotide or polypeptide in the sequence listing. It is recognized that almost daily, new discoveries are announced for gene variants, particularly for those genes under intense study, such as genes implicated in diseases like cancer. Therefore, the sequence listings given are exemplary of what is now reported for a gene, but it recognized that for the purpose of an analytical methodology, variants of the gene, and their fragments are also included.

One embodiment of what is disclosed is a panel of biomarkers with the selectivity and sensitivity required for managing patient care for CRC. In Table 1, entries 1-22 are the polynucleotide coding sequences for a panel of biomarkers, and include the name and abbreviation of the gene. Entries 23-44 in Table 1 are the protein, or polypeptide, amino acid sequences that correspond to the coding sequences for entries 1-22. A biomarker, as defined by the National Institutes of Health (NIH) is a molecular indicator of a specific biological property; a biochemical feature or facet that can be used to measure the progress of disease or the effects of treatment. A panel of biomarkers is a selection of biomarkers. Biomarkers may be from a variety of classes of molecules. As previously mentioned, there is still a need for biomarkers for CRC having the selectivity and sensitivity required to be effective for all aspects of patient care management. Therefore, the selection of an effective set of biomarkers is differentiating in providing the basis for effective determination of CRC.

In another embodiment of this disclosure, expression levels of polynucleotides for the biomarkers indicated in SEQ ID NOs 1-22, are used in the determination of CRC. Such analysis of polynucleotide expression levels is frequently referred to in the art as gene expression profiling. In gene expression profiling, levels of mRNA in a sample are measured as a leading indicator of a biological state, in this case, as an indicator of CRC. One of the most common methods for analyzing gene expression profiling is to create multiple copies from mRNA in a biological sample using a process known as reverse transcription. In the process of reverse transcription, the mRNA from the sample is used to create copies of the corresponding DNA sequence from which the mRNA was originally transcribed. In the reverse transcription amplification process, copies of DNA are created without the regulatory regions in the gene known as introns. These multiple copies made from mRNA are therefore referred to as copy DNA, or cDNA. Entries 45-88 are the sets of primers used in the reverse transcription process for each gene listed in entries 1-22.

Since the reverse transcription procedure amplifies copies of cDNA proportional to the original level of mRNA in a sample, it has become a standard method that allows the analysis of even low levels of mRNA present in a biological sample. Genes may either be up regulated or down regulated in any particular biological state, and hence mRNA levels shift accordingly.

In still another embodiment of this disclosure, expression levels of proteins listed in SEQ ID NOs 23-44, which correspond to the genes indicated in SEQ ID NOs 1-22, are disclosed. The term “polypeptide” or “polypeptides” is used interchangeably with the term “protein” or “proteins” herein. As discussed previously, proteins have been long investigated for their potential as biomarkers, with limited success. There is value in protein biomarkers as complementary to polynucleotide biomarkers. Reasons for having the information provided by both types of biomarkers include the current observations that mRNA expression levels are not good predictors of protein expression levels, and that mRNA expression levels tell nothing of the post-translational modifications of proteins that are key to their biological activity. Therefore, in order to understand the expression levels of proteins, and their complete structure, the direct analysis of proteins is required.

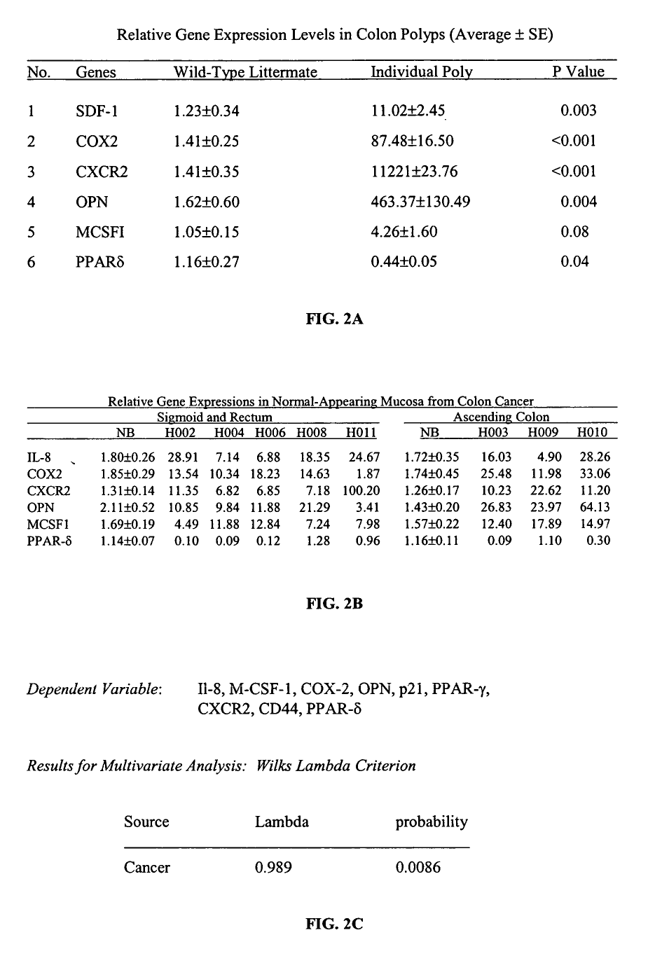

FIGS. 2A-2B show an exemplary panel of biomarkers from the list of 22 biomarkers for which gene expression levels are compared in the mouse MIN model, and in human subjects. The selection for the panel is taken from across the list of the 22 biomarkers and is taken for the purpose of easy visual assimilation of data in order to demonstrate the utility of a panel. Typically, for complex data sets represented in the 22 member panel of biomarkers, multivariate analysis (MANOVA) is applied, such as that demonstrated in FIG. 2C.

In FIG. 2A, the data reported for the mouse MIN studies represent statistical averaging of a number of animal subjects, and the standard error is reported. The p value on the right indicates the degree of confidence that the values are significantly different. As an example, the first gene listed, SDF-1, is related to the human IL-8 gene, and is in the same super family. For SDF-1, the p value of 0.003 indicates that the probability that the differences in the values of the wildtype control and that of the adenomous polyps of the MIN mice occurred by chance alone is only 3 in 1000. Screening the expression levels in adenomous polyps in the subject mice was specifically targeted, since it has been established that adenomous polyps are useful in risk assessment for CRC. What is demonstrated in FIG. 2A is that the panel of 6 clearly differentiate the results of the MIN mice over that of the wildtype control.

FIGS. 2B-2C address the issue of selectivity for biomarker panels. Regarding biomarkers that have an acceptable level of selectivity for CRC, the incidence of CRC for individuals in families with a history of CRC is 3-4 times that of the general population. However, It is now estimated that 6% of all Americans will develop CRC, and of those 70-80% will occur in people of average risk. There is clearly a need for biomarkers that have the necessary selectivity required for confidence in the determination of CRC.

In FIG. 2B, the same panel of 6 biomarkers established in the mouse MIN model in FIG. 2A are the basis for determination of CRC in human subjects. In FIG. 2B, the results of biopsy tissue determined to be normal by histological evaluation taken from patients known to have CRC are compared to biopsy tissue from individuals validated as normal controls. It should be noted that histological methodologies are the accepted standard for the identification of a cancerous colonic lesion. There are two aspects of FIG. 2B to further discuss. First, values for gene expression profiling for patient vs. normal control may vary either up, as in the case of IL 8, or down, as in the case of PPAR-δ. It is the determination of the collective shift for the patient vs. normal control that is significant when using a panel of biomarkers. Second, in glancing through the patient data, sample-to-sample variation can be noted, which is anticipated, given all the patient-to-patient variables. It is clear at a glance that the expression levels for the panel taken as a group distinguish the patient samples overall from the normal control group, even though a value for any one specific biomarker may not in itself distinguish the patient sample from the normal control. For example, the patient designated as H008 has an expression level for PPAR-δ that is not distinct from the normal control. However, at a glance it is clear that the results of the panel for H008 distinguish it from the normal control set. This demonstrates in principle why a validated panel of markers, given the complexity and variability of biology, enhance the selectivity of a determination vs. a single marker alone.

FIG. 2C further serves to emphasize the value of a panel of biomarkers in enhancing the selectivity of a determination between patient vs. normal samples. An example of demonstrating the use of MANOVA for a panel of 9 biomarkers selected from the group of 22 is demonstrated in FIG. 2C. In this study, 78 sigmoidal-rectal biopsies from 12 normal patients, and 63 sigmoidal-rectal biopsies from non-cancerous sections of 6 patients with sigmoidal-rectal carcinoma were compared. The Wilks' Lambda criterion was used to assess the difference between the patient samples and normal control samples using the 9 biomarkers listed. The lambda value close to 1.0 signifies a significant difference between the patient and normal samples is indicated, with the probability of about 9 chances in 1000 that the difference is by chance alone.

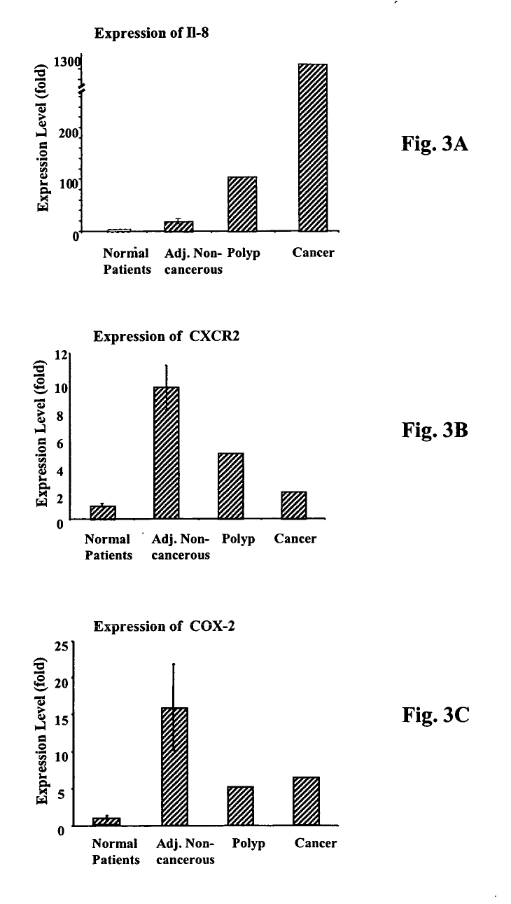

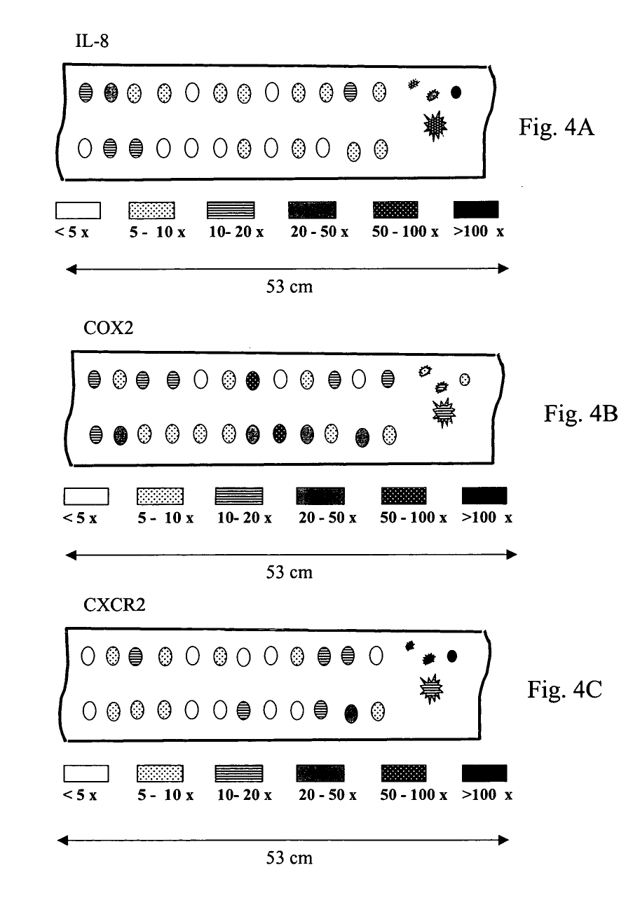

FIGS. 3A-3C and FIGS. 4A-4C address the issue of sensitivity for biomarker panels. As previously mentioned, since survival rates are greatly enhanced with the earliest indication of CRC, biomarkers for risk assessment and early detection of CRC have been long sought. The difference between risk assessment and early detection is the degree of certainty regarding acquiring CRC. Biomarkers that are used for risk assessment confer less than 100% certainty of CRC within a time interval, whereas biomarkers used for early detection confer an almost 100% certainty of the onset of the disease within a specified time interval. Risk factors may be used as surrogate end points for individuals not diagnosed with cancer, providing they there is an established relationship between the surrogate end point and a definitive outcome. An example of an established surrogate end point for CRC is the example of adenomous polyps. What has been established is that the occurrence of adenomous polyps are a necessary, but not sufficient condition for an individual to later develop CRC. This is demonstrated by the fact that 90% percent of all preinvasive cancerous lesions are adenomous polyps or precursors, but not all individuals with adenomous polyps go on to later develop CRC.

FIGS. 3A-3C show graphs of gene expression levels taken for multiple biopsy samples taken from the colon of one exemplary patient diagnosed with CRC. The determination of cancerous lesions, polyps, and adjacent tissues was made by conventional histological methods. The expression levels for three of the panel of biomarkers are shown for the biopsy samples categorized in that fashion. Again, as was demonstrated with the examples given in FIGS. 2A-2C, it is evident that the three markers taken together for the cancerous lesions sampled are significantly different than the normal controls, even though one by itself (CXCR2) would not have been differentiating for this patient. What is additionally indicated in this representation is the distinction between the results of the polyp vs. the normal control. Given that polyps are already accepted as surrogate endpoints for CRC, then a determination of the presence of polyps by a validated analytical methodology using a minimally invasive method, such as a swab, or a non-invasive sampling method, such as a stool sample, would also serve as surrogate end point for risk assessment.

FIGS. 4A-4C show the results of gene expression levels for three of the biomarkers in biopsy samples taken over a 53 cm region of the colon of a patient with CRC. The irregularly shaped objects represent biopsy samples that were confirmed to be cancerous lesions by histological methodology, while the oval shapes represent samples that were determined to be non-cancerous by histological methodology. Gene expression profiling was done for each of the biopsy samples, as well. The results of the expression profiling, where the legend indicates relative levels in the patient biopsy samples as compared to normal controls, are depicted in FIGS. 4A-4C.

The representation of FIGS. 4A-4C indicates the distance over which the biomarkers are able to distinguish differences in the colon tissue for the patient, where these biopsy samples were rendered normal by conventional histological analysis. These results demonstrate that it is possible to sample cells through a minimally invasive swabbing collection method from an area distant from a cancerous lesion, but capable of indicating a non-normal colon condition. Moreover, collection of a stool sample is an already validated sampling method for collecting sloughed cells or cell debris from which these determinations may be made. In that regard, samples taken either minimally invasively or non-invasively would render samples that could be analyzed using the disclosed panel of biomarkers. Such non-invasive procedures not only reduce the cost of determination of CRC, but reduce the discomfort and risk associated with current methodology. All these factors together increase the attractiveness of regular testing, and hence patient compliance. Increased patient compliance, coupled with an effective determination for CRC, enhance the prospects for early detection, and enhanced survival rates.

Methods and kits for the polynucleotide and polypeptide expression profiling for the panel of molecular markers are also contemplated as part of the present disclosure.

In one embodiment, a method for gene expression profiling comprises measuring cDNA levels for biomarkers selected in the claimed panel. Such a method requires the use of primers, enzymes, and other reagents for the preparation, detection, and quantitation of cDNAs. The method of creating cDNA from mRNA in a sample is referred to as the reverse transcriptase polymer chain reaction (RT-PCR). The primers listed in SEQ ID NOs 45-88 are particularly suited for use in gene expression profiling using RT-PCR based on the claimed panel. A series of primers were designed using Primer Express Software (Applied Biosystems, Foster City, Calif.). Specific candidates were chosen, and then tested to verify that only cDNA was amplified, and not contaminated by genomic DNA. The primers listed in SEQ ID NOs 45-88 were specifically designed, selected, and tested accordingly. In addition to the primers, reagents such as one including a dinucleotide triphosphate mixture having all four dinucleotide triphosphates (e.g. dATP, dGTP, dCTP, and dTTP), one having the reverse transcriptase enzyme, and one having a thermostable DNA polymerase are required for RT-PCR. Additionally buffers, inhibitors and activators are also required for the RT-PCR process. Once the cDNA has been sufficiently amplified to a specified end point, the cDNA sample must be prepared for detection and quantitation. Though a number of detection schemes are contemplated, as will be discussed in more detail below, one method contemplated for detection of polynucleotides is fluorescence spectroscopy, and therefore chromophores that are suited to fluorescence spectroscopy are desirable for labeling polynucleotides. One example of such a fluorescent label is SYBR Green, though numerous related chromophores exist, and are known in the art.

In another embodiment, a method for protein expression profiling comprises using an antibody panel based on the claimed panel of biomarkers for measuring targeted polypeptide levels from a biological sample. In one embodiment contemplated for the method, the antibodies for the panel are bound to a solid support. The method for protein expression profiling may use a second antibody having specificity to some portion of the bound polypeptide. Such a second antibody may be labeled with molecules useful for detection and quantitation of the bound polypeptides, and therefore in binding to the polypeptide label it for detection and quantitation. Additionally, other reagents are contemplated for labeling the bound polypeptides for detection and quantitation. Such reagents may either directly label the bound polypeptide or, analogous to a second antibody, may be a moiety with specificity for the bound polypeptide having labels. Examples of such moieties include but are not limited to small molecules such as cofactors, substrates, complexing agents, and the like, or large molecules, such as lectins, peptides, olionucleotides, and the like. Such moieties may be either naturally occurring or synthetic.

Examples of detection modes contemplated for the disclosed methods include, but are not limited to spectroscopic techniques, such as fluorescence and UV-Vis spectroscopy, scintillation counting, and mass spectroscopy. Complementary to these modes of detection, examples of labels for the purpose of detection and quantitation used in these methods include, but are not limited to chromophoric labels, scintillation labels, and mass labels. The expression levels of polynucleotides and polypeptides measured using these methods may be normalized to a control established for the purpose of the targeted determination. These methods are believed useful in providing determinations as the basis of effective management of patient care for CRC. These methods may also be used in the discovery of therapeutic interventions for CRC. Additionally, not only biopsy samples from sigmoidoscopy, colonoscopy, or surgery may be analyzed by these methods, but biological samples from non-invasive or minimally evasive collection methods are indicated for these methods, as well.

It is further contemplated in what is disclosed to provide kits having the reagents and procedures that facilitate the ready implementation of the methods, and provide consistency and quality control thereby.

In one embodiment, a kit for gene expression profiling comprises the reagents and instructions necessary for the gene expression profiling of the claimed panel. Thus, for example, the reagents may include primers, enzymes, and other reagents for the preparation, detection, and quantitation of cDNAs for the claimed panel of biomarkers. As discussed above, the method of creating cDNA from mRNA in a sample is referred to as the reverse transcriptase polymer chain reaction (RT-PCR). The primers listed in SEQ ID NOs 45-88 are particularly suited for use in gene expression profiling using RT-PCR based on the claimed panel. The primers listed in SEQ ID NOs 45-88 were specifically designed, selected, and tested accordingly. In addition to the primers, reagents such as one including a dinucleotide triphosphate mixture having all four dinucleotide triphosphates (e.g. dATP, dGTP, dCTP, and dTTP), one having the reverse transcriptase enzyme, and one having a thermostable DNA polymerase are required for RT-PCR. Additionally buffers, inhibitors and activators used for the RT-PCR process are suitable reagents for inclusion in the kit embodiment. Once the cDNA has been sufficiently amplified to a specified end point, the cDNA sample must be prepared for detection and quantitation. One method contemplated for detection of polynucleotides is fluorescence spectroscopy, and therefore chromophores that are suited to fluorescence spectroscopy are desirable for labeling polynucleotides and may also be included in reagents of the kit embodiment. Instructions included with the kit embodiment for gene expression profiling preferably teach the user the following steps: to obtain a biological sample; to isolate cellular RNA from the sample; to amplify copies of cDNA from the sample for each biomarker in the panel, and the panel for which the reagents are provided; and to quantify levels of cDNA amplified from the sample. Though tissue samples from a variety of procedures may be used, the instructions for obtaining a biological sample are preferably whereby the user obtains a sample of colorectal cells in a minimally invasive manner, such as by use of a swab or collection of a stool sample. The instructions may also preferably include the step of comparing the cDNA levels quantified to a control.

In another embodiment, a kit for protein expression profiling comprises the reagents and instructions necessary for protein expression profiling of the claimed panel. Thus, in this embodiment, the kit for protein expression profiling includes supplying an antibody panel based on the claimed panel of biomarkers for measuring targeted polypeptide levels from a biological sample. One embodiment contemplated for such a panel includes the antibody panel bound to a solid support. Additionally, the reagents included with the kit for protein expression profiling may use a second antibody having specificity to some portion of the bound polypeptide. Such a second antibody may be labeled with molecules useful for detection and quantitation of the bound polypeptides, and therefore in binding to the polypeptide label it for detection and quantitation. Additionally, other reagents are contemplated for labeling the bound polypeptides for detection and quantitation. Such reagents may either directly label the bound polypeptide or, analogous to a second antibody, may be a moiety with specificity for the bound polypeptide having labels. Examples of such moieties include but are not limited to small molecules such as cofactors, substrates, complexing agents, and the like, or large molecules, such as lectins, peptides, olionucleotides, and the like. Such moieties may be either naturally occurring or synthetic. Instructions for the protein expression profiling kit preferably teach the user: to obtain a biological sample; to use the antibody panel supplied with the kit for each biomarker in the panel to bind the polypeptides from the sample; and to quantify levels of polypeptides bound from the sample to the antibody panel. Preferably, the kit instructions also include a step of comparing the polypeptide levels to a control. Preferably the biological sample is obtained by a minimally invasive procedure such as use of a swab to through a stool sample.

Additionally, consumable labware required for sample collection, preparation, and analysis may be provided with the kits.

What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

1. A panel of biomarkers for colorectal cancer and colorectal polyps comprising at least two polynucleotides selected from SEQ ID NOs 1-5.

2. The panel of claim 1, where the panel is selected for analysis of polynucleotide expression levels for colorectal cancer and colorectal polyps.

3. The panel of claim 2, where the polynucleotide expression levels are mRNAs.

4. The panel of claim 2, where the polynucleotide expression levels are cDNAs.

5. The panel of claim 1, where at least one of the polynucleotides is a fragment.

6. The panel of claim 1, where at least one of the polynucleotides is a variant.

7. The panel of claim 1, where the panel is used for the management of patient care in colorectal cancer and colorectal polyps.

8. The panel of claim 7, where the management of patient care includes one or more of risk assessment, early diagnosis, establishing prognosis, monitoring patient treatment, and detecting relapse.

9. The panel of claim 1, where the panel is used in discovery of therapeutic intervention of colorectal cancer and colorectal polyps.

10. A panel of biomarkers for colorectal cancer and colorectal polyps comprising:
at least two polynucleotides selected from SEQ ID NOs 1-5; and
at least one polynucleotide selected from SEQ ID NOs 6-14.

11. The panel of claim 10, where the panel is selected for analysis of polynucleotide expression levels for colorectal cancer and colorectal polyps.

12. The panel of claim 11, where the polynucleotide expression levels are mRNAs.

13. The panel of claim 11, where the polynucleotide expression levels are cDNAs.

14. The panel of claim 10, where at least one of the polynucleotides is a fragment.

15. The panel of claim 10, where at least one of the polynucleotides is a variant.

16. The panel of claim 10, where the panel is used in the management of patient care for colorectal cancer and colorectal polyps.

17. The panel of claim 16, where the management of patient care includes one or more of risk assessment, early diagnosis, establishing prognosis, monitoring patient treatment, and detecting relapse.

18. The panel of claim 10, where the panel is used in discovery of therapeutic intervention of colorectal cancer and colorectal polyps.

19. A panel of biomarkers for colorectal cancer and colorectal polyps comprising:
at least two polynucleotides selected from SEQ ID NOs 1-5;
at least one polynucleotide selected from SEQ ID NOs 6-14; and
at least one polynucleotide selected from SEQ ID NOs 15-22.

20. The panel of claim 19, where the panel is selected for analysis of polynucleotide expression levels for colorectal cancer and colorectal polyps.

21. The panel of claim 20, where the polynucleotide expression levels are mRNAs.

22. The panel of claim 20, where the polynucleotide expression levels are cDNAs.

23. The panel of claim 19, where at least one of the polynucleotides is a fragment.

24. The panel of claim 19, where at least one of the polynucleotides is a variant.

25. The panel of claim 25, where the panel is the basis for management of patient care in colorectal cancer and colorectal polyps.

26. The panel of claim 19, where the management of patient care includes one or more of risk assessment, early diagnosis, establishing prognosis, monitoring patient treatment, and detecting relapse.

27. The panel of claim 25, where the panel is used in discovery of therapeutic intervention of colorectal cancer and colorectal polyps.

28. A panel of biomarkers for colorectal cancer and colorectal polyps comprising at least two polypeptides selected from SEQ ID NOs 23-27.

29. The panel of claim 28, where the panel is selected for analysis of polypeptide expression levels for colorectal cancer and colorectal polyps.

30. The panel of claim 28, where at least one of the polypeptides is a fragment.

31. The panel of claim 28, where at least one of the polypeptides is a variant.

32. The panel of claim 28, where the panel is used in the management of patient care in colorectal cancer and colorectal polyps.

33. The panel of claim 32, where the management of patient care includes one or more of risk assessment, early diagnosis, establishing prognosis, monitoring patient treatment, and detecting relapse.

34. The panel of claim 28, where the panel is used in discovery of therapeutic intervention of colorectal cancer and colorectal polyps.

35. A panel of biomarkers for colorectal cancer and colorectal polyps comprising:
at least two polypeptides selected from SEQ ID NOs 23-27; and
at least one polypeptide selected from SEQ ID NOs 28-36.

36. The panel of claim 35, where the panel is selected for analysis of polypeptide expression levels for colorectal cancer and colorectal polyps.

37. The panel of claim 35, where at least one of the polypeptides is a fragment.

38. The panel of claim 35, where at least one of the polypeptides is a variant.

39. The panel of claim 35, where the panel is used in the management of patient care in colorectal cancer and colorectal polyps.

40. The panel of claim 39, where the management of patient care includes one or more of risk assessment, early diagnosis, establishing prognosis, monitoring patient treatment, and detecting relapse.

41. The panel of claim 35, where the panel is used in discovery of therapeutic intervention of colorectal cancer and colorectal polyps.

42. A panel of biomarkers for colorectal cancer and colorectal polyps comprising:
at least two polypeptides selected from SEQ ID NOs 23-27;
at least one polypeptide selected from SEQ ID NOs 28-36; and
at least one polypeptide selected from SEQ ID NOs 37-44.

43. The panel of claim 42, where the panel is selected for analysis of polypeptide expression levels for colorectal cancer and colorectal polyps.

44. The panel of claim 42, where at least one of the polypeptides is a fragment.

45. The panel of claim 42, where at least one of the polypeptides is a variant.

46. The panel of claim 42, where the panel is used in the management of patient care in colorectal cancer and colorectal polyps.

47. The panel of claim 46, where the management of patient care includes one or more of risk assessment, early diagnosis, establishing prognosis, monitoring patient treatment, and detecting relapse.

48. The panel of claim 42, where the panel is used in discovery of therapeutic intervention of colorectal cancer and colorectal polyps.

49. A method for measuring expression levels of polynucleotides from biomarkers for colorectal cancer and colorectal polyps, comprising:
selecting a panel of biomarkers comprising at least two polynucleotides from SEQ ID NOs 1-5;
obtaining a biological sample;
isolating cellular RNA from the sample;
amplifying copies of cDNA from the sample for each biomarker in the panel; and
quantifying levels of cDNA amplified from the sample.

50. The method of claim 49, where the step of selecting a panel of biomarkers further comprises at least one polynucleotide from SEQ ID NOs 6-14.

51. The method of claim 49, where the step of selecting a panel of biomarkers further comprises:
at least one polynucleotide from SEQ ID NOs 6-14; and
at least one polynucleotide from SEQ ID NOs 15-22.

52. The method of claim 49, where the step of amplifying copies of cDNA further comprises at least two sets of primers chosen from SEQ. ID NOs 45-50.

53. The method of claim 52, where the step of amplifying copies of cDNA further comprises using enzymes and reagents for the preparation of cDNAs.

54. The method of claim 49, where the step of quantifying the levels of cDNA further comprises labeling cDNA.

55. The method of claim 54, where labeling cDNA includes at least one chromophore.

56. The method of claim 49, where the cDNA levels for the sample are compared to a control.

57. The method of claim 56, where the comparison is used in the management of patient care in colorectal cancer and colorectal polyps.

58. The method of claim 57, where the management of patient care includes one or more of risk assessment, early diagnosis, establishing prognosis, monitoring patient treatment, and detecting relapse.

59. The method of claim 56, where the comparison is used in discovery of therapeutic intervention of colorectal cancer and colorectal polyps.

60. The method of claim 49, where the step of obtaining a biological sample is by obtaining a sample of colorectal cells.

61. The method of claim 60, where the step of obtaining a sample of colorectal cells is minimally invasive.

62. The method of claim 61, where the minimally invasive step is by use of a swab.

63. The method of claim 60, where the step of obtaining a sample of colorectal cells is non-invasive.

64. The method of claim 63, where the non-invasive step is by collection of a stool sample.

65. A method for measuring expression levels of polypeptides from biomarkers for colorectal cancer and colorectal cancer, comprising:
selecting a panel of biomarkers comprising at least two polypeptides from SEQ ID NOs 23-27;
obtaining a biological sample;
creating an antibody panel for each biomarker in the panel;
using the antibody panel to bind the polypeptides from the sample; and
quantifying levels of polypeptides bound from the sample to the antibody panel.

66. The method of claim 65, where the step of selecting a panel of biomarkers further comprises at least one polypeptide from SEQ ID NOs 28-36.

67. The method of claim 65, where the step of selecting a panel of biomarkers further comprises:
at least one polypeptide from SEQ ID NOs 28-36; and
gat least one polypeptide from SEQ ID NOs 37-44.

68. The method of claim 65, where the polypeptide levels for the sample are compared to a control.

69. The method of claim 68, where the comparison is used in the management of patient care in colorectal cancer and colorectal polyps.

70. The method of claim 69, where the management of patient care includes one or more of risk assessment, early diagnosis, establishing prognosis, monitoring patient treatment, and detecting relapse.

71. The method of claim 68, where the comparison is used in discovery of therapeutic intervention of colorectal cancer and colorectal polyps.

72. The method of claim 65, where the step of obtaining a biological sample is by obtaining a sample of colorectal cells.

73. The method of claim 72, where the step of obtaining a sample of colorectal cells is minimally invasive.

74. The method of claim 73, where the minimally invasive step is by use of a swab.

75. The method of claim 72, where the step of obtaining a sample of colorectal cells is non-invasive.

76. The method of claim 75, where the non-invasive step is by collection of a stool sample.

77. The method of claim 65, where the step of quantifying the bound polypeptides further comprises labeling the polypeptides.

78. The method of claim 77, where labeling the polypeptides comprises using a second antibody.

79. A kit for the determination of colorectal cancer and colorectal polyps comprising:
at least one reagent that is used in analysis of polynucleotide expression levels for a panel of biomarkers for colorectal cancer and colorectal polyps, where the panel comprises at least two polynucleotides listed in SEQ ID NOs 1-5; and
instructions for using the kit for analyzing the expression levels.

80. The kit of claim 79, where the panel of biomarkers further comprises at least one polynucleotides listed in SEQ ID NOs 6-14.

81. The kit of claim 79, where the panel of biomarkers further comprises:
at least one polynucleotide selected from SEQ ID NOs 6-14; and
at least one polynucleotide selected from SEQ ID NOs 15-22.

82. The kit of claim 79, where the polynucleotide expression levels are mRNAs.

83. The kit of claim 79, where the polynucleotide expression levels are cDNAs.

84. The kit of claim 83, where the reagent comprises at least two sets of primers chosen from SEQ. ID NOs 45-50.

85. The kit of claim 84, further comprising reagents for the preparation of cDNA.

86. The kit of claim 79, comprising a reagent that is used for detection and quantitation of polynucleotides.

87. The kit of claim 86, where the reagent includes at least one chromophore.

88. The kit of claim 79, further comprising consumable labware for at least one of sample collection, sample preparation, and sample analysis.

89. A kit for the determination of colorectal cancer and colorectal polyps comprising:
at least one reagent used in that analysis of polypeptide expression levels for a panel of biomarkers for colorectal cancer and colorectal polyps, where the panel comprises at least two polypeptides listed in SEQ. ID NOs 23-27; and
instructions for using the kit for analyzing the expression levels.

90. The kit of claim 89, where the panel of biomarkers further comprises at least one polynucleotides listed in SEQ ID NOs 28-36.

91. The kit of claim 89, where the panel of biomarkers further comprises:
at least one polynucleotide selected from SEQ ID NOs 28-36; and
at least one polynucleotide selected from SEQ ID NOs 37-44.

92. The kit of claim 89, where the reagent is an antibody reagent that binds a polypeptide selected in the panel.

93. The kit of claim 89, further comprising a reagent that is used for detection and quantitation of a bound polypeptide.

94. The kit of claim 93, where the reagent includes a second antibody.

95. The kit of claim 89, further comprising consumable labware for at least one of sample collection, sample preparation, and sample analysis.


SEQUENCE LISTING (88 sequences)

SEQ ID NO: 1 - DNA, 1629 bp, Homo sapiens
  CDS (74)..(274)
gcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattatgtaaagctttctgatggaagagagctctgtctggaccccaaggaaaactgggtgcagagggttgtggagaagtttttgaagagggctgagaattcagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagt

SEQ ID NO: 2 - DNA, 3356 bp, Homo sapiens
  CDS (98)..(1909)
gtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagatta

SEQ ID NO: 3 - DNA, 1750 bp, Homo sapiens
  CDS (53)..(1132)
cctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctttgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagcagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacgggatgaaaccctgtctctactaaaaatacaaaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggt

SEQ ID NO: 4 - DNA, 597 bp, Homo sapiens
  CDS (1)..(597)
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatatctgggcctccctgaaaaccacatcgtcttccctgtcccaatcgaccagtgtatcgacggctga

SEQ ID NO: 5 - DNA, 369 bp, Homo sapiens
  CDS (1)..(369)
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 6 - DNA, 3939 bp, Homo sapiens
  CDS (106)..(1767)
cctgggtcctctcggcgccagagccgctctccgcatcccaggacagcggtgcggccctcggccggggcgcccactccgcagcagccagcgagccagctgccccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacaccgccaatcccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactgctacagccttgcccagggtgggccccgtgatgcccactggccaggactggaatcacaccccccagaagacagaccatccatctgccctgctcagagaccccccggagccaggctctcccaggatctcatcactgcgcccccaggccctcagcaacccctccaccctctctgctcagccacagctttccagaagccactcctcgggcagcgtgctgccccttggggagctggagggcaggaggagcaccagggatcggacgagccccgcagagccagaagcagcaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggctgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctcttcgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcagcctcctctcactcccttccatgccggaacccaggccagggacccaccggcctgtggtttgtgggaaagcagggtgcacgctgaggagtgaaacaaccctgcacccagagggcctgcctggtgccaaggtatcccagcctggacaggcatggacctgtctccagacagaggagcctgaagttcgtggggcgggacagcctcggcctgatttcccgtaaaggtgtgcagcctgagagacgggaagaggaggcctctgcacctgctggtctgcactgacagcctgaagggtctacaccctcggctcacctaagtccctgtgctggttgccaggcccagaggggaggccagccctgccctcaggacctgcctgacctgccagtgatgccaagagggggatcaagcactggcctctgcccctcctccttccagcacctgccagagcttctccagcaggccaagcagaggctcccctcatgaaggaagccattgcactgtgaacactgtacctgcctgctgaacagcctccccccgtccatccatgagccagcatccgtccgtcctccactctccagcctctccccagcctcctgcactgagctggcctcaccagtcgactgagggagcccctcagccctgaccttctcctgacctggcctttgactccccggagtggagtggggtgggagaacctcctgggccgccagccagagccgctctttaggctgtgttcttcgcccaggtttctgcatcttccactttgacattcccaagagggaagggactagtgggagagagcaagggaggggagggcacagacagagagcctacagggcgagctctgactgaagatgggcctttgaaatataggtatgcacctgaggttgggggagggtctgcactcccaaaccccagcgcagtgtcctttccctgctgccgacaggaacctggggctgagcaggttatccctgtcaggagccctggactgggctgcatctcagccccacctgcatggtatccagctcccatccacttctcacccttctttcctcctgaccttggtcagcagtgatgacctccaactctcacccaccccctctaccatcacctctaaccaggcaagccagggtgggagagcaatcaggagagccaggcctcagcttccaatgcctggagggcctccactttgtggccagcctgtggtgctggctctgaggcctaggcaacgagcgacagggctgccagttgcccctgggttcctttgtgctgctgtgtgcctcctctcctgccgccctttgtcctccgctaagagaccctgccctacctggccgctgggccccgtgactttcccttcctgcccaggaaagtgagggtcggctggccccaccttccctgtcctgatgccgacagcttagggaagggcactgaacttgcatatggggcttagccttctagtcacagcctctatatttgatgctagaaaacacatatttttaaatggaagaaaaataaaaaggcattcccccttcatccccctaccttaaacatataatattttaaaggtcaaaaaagcaatccaacccactgcagaagctctttttgagcacttggtggcatcagagcaggaggagccccagagccacctctggtgtcccccaggctacctgctcaggaaccccttctgttctctgagaactcaacagaggacattggctcacgcactgtgagattttgtttttatacttgcaactggtgaattattttttataaagtcatttaaatatctatttaaaagataggaagctgcttatatatttaataataaaagaagtgcacaagctgccgttgacgtagctcgag

SEQ ID NO: 7 - DNA, 1024 bp, Homo sapiens
  CDS (1)..(321)
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag

SEQ ID NO: 8 - DNA, 1064 bp, Homo sapiens
  CDS (78)..(395)
  modified_base (27): a, c, t, g, other or unknown
cacagccgggtcgcaggcacctccccngccagctctcccgcattctgcacagcttcccgacgcgtctgctgagccccatggcccacgccacgctctccgccgcccccagcaatccccggctcctgcgggtggcgctgctgctcctgctcctggtgggcagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaagggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtngggggattgaaatgcaagcaattagtggatcactgttagggtaagggaatgtatgtacacatctattttttatacttttttttttaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatg

SEQ ID NO: 9 - DNA, 1469 bp, Homo sapiens
  CDS (102)..(1001)
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaaaaaaaaaaaaaaaacaaaaaaaaaaaa

SEQ ID NO: 10 - DNA, 1256 bp, Homo sapiens
  CDS (145)..(1029)
gcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacggctccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgcctcttgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgaggggcccaggcaggcgggcgccaccgccacccgcagcgagggcggagccggccccaggtgctccacatgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagag

SEQ ID NO: 11 - DNA, 2121 bp, Homo sapiens
  CDS (559)..(1875)
ctgctcgcggccgccaccgccgggccccggccgtccctggctcccctcctgcctcgagaagggcagggcttctcagaggcttggcgggaaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgcttgatcccccaggccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaa

SEQ ID NO: 12 - DNA, 2098 bp, Homo sapiens
  CDS (79)..(570)
cctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgatatacagcaggtgctcaataaacgattcttagtg

SEQ ID NO: 13 - DNA, 827 bp, Homo sapiens
  CDS (256)..(570)
ggcacgaggcaaagggcggcgcagcggctgccgagctcggccctggaggcggcgagaacatggtgcgcaggttcttggtgaccctccggattcggcgcgcgtgcggcccgccgcgagtgagggttttcgtggttcacatctcgtggttcacgggggagtgggcagcgccaggggcgcccgccgctgtggccctcgtgctgatgctactgaggagccagcgtctagggcagcagccgcttcctagaagaccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctccggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaaaaaaaaaaaaaaaaaaaa

SEQ ID NO: 14 - DNA, 1275 bp, Homo sapiens
  CDS (163)..(681)
cctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgcgcaggttcttggtgaccctccggattcggcgcgcgtgcggcccgccgcgagtgagggttttcgtggttcacatcccgcggctcacgggggagtgggcagcgccaggggcgcccgccgctgtggccctcgtgctgatgctactgaggagccagcgtctagggcagcagccgcttcctagaagaccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactc

SEQ ID NO: 15 - DNA, 1850 bp, Homo sapiens
  CDS (213)..(1616)
ggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagcttggagctcggcgcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggaggtggctaatctcaggactgggaag

SEQ ID NO: 16 - DNA, 1609 bp, Homo sapiens
  CDS (92)..(1606)
ttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 17 - DNA, 3301 bp, Homo sapiens
  CDS (338)..(1660)
  modified_base (2966)..(2972): a, c, t, g, other, or unknown
gaattctgcggagcctgcgggacggcggcgggttggcccgtaggcagccgggacagtgttgtacagtgttttgggcatgcacgtgatactcacacagtggcttctgctcaccaacagatgaagacagatgcaccaacgagggtctggaatggtctggagtggtctggaaagcagggtcagatacccctggaaaactgaagcccgtggagcaatgatctctacaggactgcttcaaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagctctcttcctgtctttgttgtctccctctttctcagttcctctttcttttctaattcctgttgctctgtttcttcctttctgtaggtttctctcttcccttctcccttctcccttgccctccctttctctctcctatccccacgtctgtcctcctttcttattctgtgagatgttttgtattatttcaccagcagcatagaacaggacctctgcttttgcacaccttttccccaggagcagaagagagtgggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtggcagaccccggcagtagagccgagatgcctccccaagactgtcattgcccctccgatcgtgaggccacccactgacccaatgatcctctccagcagcacacctcagccccactgacacccagtgtccttccatcttcacactggtttgccaggccaatgttgctgatggcccctccagcacacacacataagcactgaaatcactttacctgcaggcaccatgcacctcccttccctccctgaggcaggtgagaacccagagagaggggcctgcaggtgagcaggcagggctgggccaggtctccggggaggcaggggtcctgcaggtcctggtgggtcagcccagcacctcgcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctatgtgactctgggtggaagtgcccagcccctgcctgacggnnnnnnngatcactctctgctggcaggattcttcccgctccccacctacccagctgatgggggttggggtgcttctttcagccaaggctatgaagggacagctgctgggacccacctccccccttccccggccacatgccgcgtccctgcccccacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaaaaaaaaaagcggccgcgaattc

SEQ ID NO: 18 - DNA, 3083 bp, Homo sapiens
  CDS (162)..(2387)
aaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttc

SEQ ID NO: 19 - DNA, 2539 bp, Homo sapiens
  CDS (321)..(1787)
ggagtctcttgctctggttcttgctgttcctgctcctgctcccgccgctccccgtcctgctcgcggacccaggggcgcccacgccagtgaatccctgttgttactatccatgccagcaccagggcatctgtgtccgcttcggccttgaccgctaccagtgtgactgcacccgcacgggctattccggccccaactgcaccatccctggcctgtggacctggctccggaattcactgcggcccagcccctctttcacccacttcctgctcactcacgggcgctggttctgggagtttgtcaatgccaccttcatccgagagatgctcatgcgcctggtactcacagtgcgctccaaccttatccccagtccccccacctacaactcagcacatgactacatcagctgggagtctttctccaacgtgagctattacactcgtattctgccctctgtgcctaaagattgccccacacccatgggaaccaaagggaagaagcagttgccagatgcccagctcctggcccgccgcttcctgctcaggaggaagttcatacctgacccccaaggcaccaacctcatgtttgccttctttgcacaacacttcacccaccagttcttcaaaacttctggcaagatgggtcctggcttcaccaaggccttgggccatggggtagacctcggccacatttatggagacaatctggagcgtcagtatcaactgcggctctttaaggatgggaaactcaagtaccaggtgctggatggagaaatgtacccgccctcggtagaagaggcgcctgtgttgatgcactacccccgaggcatcccgccccagagccagatggctgtgggccaggaggtgtttgggctgcttcctgggctcatgctgtatgccacgctctggctacgtgagcacaaccgtgtgtgtgacctgctgaaggctgagcaccccacctggggcgatgagcagcttttccagacgacccgcctcatcctcataggggagaccatcaagattgtcatcgaggagtacgtgcagcagctgagtggctatttcctgcagctgaaatttgacccagagctgctgttcggtgtccagttccaataccgcaaccgcattgccatggagttcaaccatctctaccactggcaccccctcatgcctgactccttcaaggtgggctcccaggagtacagctacgagcagttcttgttcaacacctccatgttggtggactatggggttgaggccctggtggatgccttctctcgccagattgctggccggatcggtgggggcaggaacatggaccaccacatcctgcatgtggctgtggatgtcatcagggagtctcgggagatgcggctgcagcccttcaatgagtaccgcaagaggtttggcatgaaaccctacacctccttccaggagctcgtaggagagaaggagatggcagcagagttggaggaattgtatggagacattgatgcgttggagttctaccctggactgcttcttgaaaagtgccatccaaactctatctttggggagagtatgatagagattggggctcccttttccctcaagggtctcctagggaatcccatctgttctccggagtactggaagccgagcacatttggcggcgaggtgggctttaacattgtcaagacggccacactgaagaagctggtctgcctcaacaccaagacctgtccctacgtttccttccgtgtgccggatgccagtcaggatgatgggcctgctgtggagcgaccatccacagagctctgaggggcaggaaagcagcattctggaggggagagctttgtgcttgtcattccagagtgctgaggccagggctgatggtcttaaatgctcattttctggtttggcatggtgagtgttggggttgacatttagaactttaagtctcacccattatctggaatattgtgattctgtttattcttccagaatgctgaactccttgttagcccttcagattgttaggagtggttctcatttggtctgccagaatactgggttcttagttgacaacctagaatgtcagatttctggttgatttgtaacacagtcattctaggatgtggagctactgatgaaatctgctagaaagttagggggttcttattttgcattccagaatcttgactttctgattggtgattcaaagtgttgtgttcctggctgatgatccagaacagtggctcgtatcccaaatctgtcagcatctggctgtctagaatgtggatttgattcattttcctgttcagtgagatatcatagagacggagatcctaaggtccaacaagaatgcattccctgaatctgtgcctgcactgagagggcaaggaagtggggtgttcttcttgggacccccactaagaccctggtctgaggatgtagagagaacaggtgggctgtattcacgccattggttggaagctaccagagctctatccccatccaggtcttgactcatggcagctgtttctcatgaagctaataaaattcgc

SEQ ID NO: 20 - DNA, 1875 bp, Homo sapiens
  CDS (232)..(519)
ggccggtgctgcgctcctctaattgggactccgagccggggctatttctggcgctggccgggctccaagaaggcatccgcatttgctaccagcggcggccgcggcggagccaggccggtcctcagcgcccagcaccgccgctcccggcaacccggagcgcgcaccgcagccggcggccgagctcgcgcatcccagccatcactcttccacctgctccttagagaagggaagatgagtgagtcgagctcgaagtccagccagcccttggcctccaagcaggaaaaggacggcactgagaagcggggccggggcaggccgcgcaagcagcctccgaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtgacccatgcgtgccgcctgctcctcactggaggagcagcttccttctgggactggacagctttgctccgctcccaccgcccccgccccttccccaggcccaccatcaccaccgcctctggccgccacccccatcttccacctgtgccctcaccaccacactacacagcacaccagccgctgcagggctcccatgggctgagtggggagcagttttcccctggcctcagttcccagctccccccgcccacccacgcatacacacatgccctcctggacaaggctaacatcccacttagccgcaccctgcacctgctgcgtccccactcccttggtggtggggacattgctctctgggcttttggtttgggggcgccctctctgcctccttcactgttccctctggcttcccatagtggggcctgggagggttccccctggccttaaaaggggcccaagcccatctcatcctggcacgccctactccactgccctggcagcagcaggtgtggccaatggaggggggtgctggcccccaggattcccccagccaaactgtctttgtcaccacgtggggctcacttttcatccttccccaacttccctagtccccgtactaggttggacagcccccttcggctacaggaaggcaggaggggtgagtcccctactccctcttcactgtggccacagcccccttgccctccgcctgggatctgagtacatattgtggtgatggagatgcagtcacttattgtccaggtgaggcccaagagccctgtggccgccacctgaggtgggctggggctgctcccctaaccctactttgcttccgccactcagccatttccccctcctcagatggggcaccaataacaaggagctcaccctgcccgctcccaacccccctcctgctcctccctgccccccaaggttctggttccatttttcctctgttcacaaactacctctggacagttgtgttgttttttgttcaatgttccattcttcgacatccgtcattgctgctgctaccagcgccaaatgttcatcctcattgcctcctgttctgcccacgatcccctcccccaagatactctttgtggggaagaggggctggggcatggcaggctgggtgaccgactaccccagtcccagggaaggtgccctgcccctaggatgctgcagcagagtgagcaagggggcccgaatcgaccataaagggtgtaggggccacctcctccccctgttctgttggggaggggtagccatgatttgtcccagcctggggctccctctctggtttcctatttacagttacttgaataaaaaaaatatccttttctggaaaaaaaaa

SEQ ID NO: 21 - DNA, 626 bp, Homo sapiens
  CDS (96)..(332)
agtctccggcgagttgttgcctgggctggacgtggttttgtctgctgcgcccgctcttcgcgctctcgtttcattttctgcagcgcgccacgaggatggcccacaagcagatctactactcggacaagtacttcgacgaacactacgagtaccggcatgttatgttacccagagaactttccaaacaagtacctaaaactcatctgatgtctgaagaggagtggaggagacttggtgtccaacagagtctaggctgggttcattacatgattcatgagccagaaccacatattcttctctttagacgacctcttccaaaagatcaacaaaaatgaagtttatctggggatcgtcaaatctttttcaaatttaatgtatatgtgtatataaggtagtattcagtgaatacttgagaaatgtacaaatctttcatccatacctgtgcatgagctgtattcttcacagcaacagagctcagttaaatgcaactgcaagtaggttactgtaagatgtttaagataaaagttcttccagtcagtttttctcttaagtgcctgtttgagtttactgaaacagtttacttttgttcaataaagtttgtatgttgcatttaaaaaaaaaaaaaa

SEQ ID NO: 22 - DNA, 3480 bp, Homo sapiens
  CDS (268)..(2922)
ggcggagatcgcgtctctttcgctccgtgtccgctgctgctcctgtgagcgcccggcgagtccgtcccgtccaccgtccgcagctggtagccagcctgcccctcgcctcgactccctttcaccaacaccgacacccacattgacacctccagtccggccagccgctccactcgttgcctttgcatctccacacatggcgtcctcgcgcagagcggcggctcctccgggggacccgcggtccccaccgtgcagcggggcatcatcaagatggtcctctcagggtgcgccatcattgtccgaggtcagcctcgtggtgggcctcctcctgagcggcagatcaacctcagcaacattcgtgctggaaatcttgctcgccgggcagccgccacacaacctgatgcaaaggatacccctgatgagccctgggcatttccagctcgagagttccttcgaaagaagctgattgggaaggaagtctgtttcacgatagaaaacaagactccccaggggcgagagtatggcatgatctaccttggaaaagataccaatggggaaaacattgcagaatcactggttgcagagggcttagccacccggagagaaggcatgagagctaataatcctgagcagaaccggctttcagaatgtgaagaacaagcaaaggcagccaagaaagggatgtggagtgaggggaacggttcacatactatccgggatctcaagtataccattgaaaacccaaggcactttgtggactcacaccaccagaagcctgttaatgctatcatcgagcatgtgcgggacggcagtgtggtcagggccctgctcctcccagattactacctggttacagtcatgctgtcaggcatcaagtgcccaacttttcgacgggaagcagatggcagtgaaactccagagccttttgctgcagaagccaaatttttcactgagtcgcgactgcttcagagagatgttcagatcattctggagagctgccacaaccagaacattgtgggtaccatccttcatccaaatggcaacatcacagagctcctcctgaaggaaggtttcgcacgctgtgtggactggtcgattgcagtttacacccggggcgcagaaaagctgagggcggcagagaggtttgccaaagagcgcaggctgagaatatggagagactatgtggctcccacagctaatttggaccaaaaggacaagcagtttgttgccaaggtgatgcaggttctgaatgctgatgccattgttgtgaagctgaactcaggcgattacaagacgattcacctgtccagcatccgaccaccgaggctggagggggagaacacccaggataagaacaagaaactgcgtcccctgtatgacattccttacatgtttgaggcccgggaatttcttcgaaaaaagcttattgggaagaaggtcaatgtgacggtggactacattagaccagccagcccagccacagagacagtgcctgccttttcagagcgtacctgtgccactgtcaccattggaggaataaacattgctgaggctcttgtcagcaaaggtctagccacagtgatcagataccggcaggatgatgaccagagatcatcacactacgatgaactgcttgctgcagaggccagagctattaagaatggcaaaggattgcatagcaagaaggaagtgcctatccaccgtgttgcagatatatctggggatacccaaaaagcaaagcagttcctgccttttcttcagcgggcaggtcgttctgaagctgtggtggaatacgtcttcagtggttctcgtctcaaactctatttgccaaaggaaacttgccttatcaccttcttgcttgcaggcattgaatgccccagaggagcccgaaacctcccaggcttggtgcaggaaggagagcccttcagcgaggaagctacacttttcaccaaggaactggtgctgcagcgagaggtggaggtggaggtggagagcatggacaaggccggcaactttatcggctggctgcacatcgacggtgccaacctgtccgtcctgctggtggagcacgcgctctccaaggtccacttcaccgccgaacgcagctcctactacaagtccctgctgtctgccgaggaggccgcaaagcagaagaaagagaaggtctgggcccactatgaggagcagcccgtggaggaggtgatgccagtgctggaggagaaggagcgatctgctagctacaagcccgtgtttgtgaccgagatcactgatgacctgcacttctacgtgcaggatgtggagaccggcacccagttccagaagctgatggagaacatgcgcaatgacattgccagtcacccccctgtagagggctcctatgccccccgcaggggagagttctgcattgccaaatttgtagatggagaatggtaccgtgcccgagtagagaaagtcgagtctcctgccaaaatacatgtcttctacattgactacggcaacagagaggtcctgccatccacccgcctgggtaccctatcacctgccttcagcactcgggtgctgccagctcaagccacggagtatgccttcgccttcatccaggtgccccaagatgatgatgcccgcacggacgccgtggacagcgtagttcgggatatccagaacactcagtgcctgctcaacgtggaacacctgagtgccggctgcccccatgtcaccctgcagtttgcagattccaagggcgatgtggggctgggcttggtgaaggaagggctggtcatggtggaggtgcgcaaggagaaacagttccagaaagtgatcacagaatacctgaatgcccaagagtcagccaagagcgccaggctgaacctgtggcgctatggagactttcgagctgatgatgcagacgaatttggctacagccgctaaggaggggatcgggtttggcccccagcccccgtcacgccagtccctcttcctctgccgggagggtgttttcaactccaaaccccagagaggggttgtacattgggtccagctttgcttcagtgtgtggaaatgtctcgtggggtggcatcggggctgcggggtggggaccccaaggctttctggggcagacccttgtcctctgggatgatgggcactgctatccacagtctctgccagttggttttatttggaggtttgtgggcttttttaaaaaaaaaaaagtcctcaaatcaggaagaaacatcaaagactatgtcctagtggagggagtaatcctaacacccaggctggccgccagctggcacctgcctctatcccagactgccctcgtcccagctctctgtccaactgttgattatgtgatttttctgatacgtccattctcaaatgccagtgtgttcacatcttcgctctggccagcccattctgtatttaaagctttttgaggcccaataaaatagtacgtgctgctgcagcccttattgatcaaaaaaa

SEQ ID NO: 23 - protein, 67 aa, Homo sapiens
MTSKLAVALLAAFLISAALCEGAVLPRSAKELRCQCIKTYSKPFHPKFIKELRVIESGPHCANTEIM

SEQ ID NO: 24 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 25 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 26 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKYLGLPENHIVFPVPIDQCIDG

SEQ ID NO: 27 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 28 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTANPIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTATALPRVGPVMPTGQDWNHTPQKTDHPSALLRDPPEPGSPRISSLRPQALSNPSTLSAQPQLSRSHSSGSVLPLGELEGRRSTRDRTSPAEPEAAPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 29 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 30 - protein, 106 aa, Homo sapiens
MAHATLSAAPSNPRLLRVALLLLLLVGSRRAAGASVVTELRCQCLQTLQGIHLKNIQSVNVRSPGPHCAQTEVIATLKNGKKACLNPASPMVQKIIEKILNKGSTN

SEQ ID NO: 31 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 32 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDGSIRPEELLQMELLLVNKLKWNLAAMTPHDFIEHFLSKMPEAEENKQIIRKHAQTFVASCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 33 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 34 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 35 - protein, 105 aa, Homo sapiens
MMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 36 - protein, 173 aa, Homo sapiens
MGRGRCVGPSLQLRGQEWRCSPLVPKGGAAAAELGPGGGENMVRRFLVTLRIRRACGPPRVRVFVVHIPRLTGEWAAPGAPAAVALVLMLLRSQRLGQQPLPRRPGHDDGQRPSGGAAAAPRRGAQLRRPRHSHPTRARRCPGGLPGHAGGAAPGRGAAGRARCLGPSARGPG

SEQ ID NO: 37 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 38 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 39 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 40 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 41 - protein, 489 aa, Homo sapiens
MLMRLVLTVRSNLIPSPPTYNSAHDYISWESFSNVSYYTRILPSVPKDCPTPMGTKGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKVGSQEYSYEQFLFNTSMLVDYGVEALVDAFSRQIAGRIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTE

SEQ ID NO: 42 - protein, 96 aa, Homo sapiens
MSESSSKSSQPLASKQEKDGTEKRGRGRPRKQPPKEPSEVPTPKRPRGRPKGSKNKGAAKTRKTTTTPGRKPRGRPKKLEKEEEEGISQESSEEEQ

SEQ ID NO: 43 - protein, 79 aa, Homo sapiens
MAHKQIYYSDKYFDEHYEYRHVMLPRELSKQVPKTHLMSEEEWRRLGVQQSLGWVHYMIHEPEPHILLFRRPLPKDQQK

SEQ ID NO: 44 - protein, 885 aa, Homo sapiens
MVLSGCAIIVRGQPRGGPPPERQINLSNIRAGNLARRAAATQPDAKDTPDEPWAFPAREFLRKKLIGKEVCFTIENKTPQGREYGMIYLGKDTNGENIAESLVAEGLATRREGMRANNPEQNRLSECEEQAKAAKKGMWSEGNGSHTIRDLKYTIENPRHFVDSHHQKPVNAIIEHVRDGSVVRALLLPDYYLVTVMLSGIKCPTFRREADGSETPEPFAAEAKFFTESRLLQRDVQIILESCHNQNIVGTILHPNGNITELLLKEGFARCVDWSIAVYTRGAEKLRAAERFAKERRLRIWRDYVAPTANLDQKDKQFVAKVMQVLNADAIVVKLNSGDYKTIHLSSIRPPRLEGENTQDKNKKLRPLYDIPYMFEAREFLRKKLIGKKVNVTVDYIRPASPATETVPAFSERTCATVTIGGINIAEALVSKGLATVIRYRQDDDQRSSHYDELLAAEARAIKNGKGLHSKKEVPIHRVADISGDTQKAKQFLPFLQRAGRSEAVVEYVFSGSRLKLYLPKETCLITFLLAGIECPRGARNLPGLVQEGEPFSEEATLFTKELVLQREVEVEVESMDKAGNFIGWLHIDGANLSVLLVEHALSKVHFTAERSSYYKSLLSAEEAAKQKKEKVWAHYEEQPVEEVMPVLEEKERSASYKPVFVTEITDDLHFYVQDVETGTQFQKLMENMRNDIASHPPVEGSYAPRRGEFCIAKFVDGEWYRARVEKVESPAKIHVFYIDYGNREVLPSTRLGTLSPAFSTRVLPAQATEYAFAFIQVPQDDDARTDAVDSVVRDIQNTQCLLNVEHLSAGCPHVTLQFADSKGDVGLGLVKEGLVMVEVRKEKQFQKVITEYLNAQESAKSARLNLWRYGDFRADDADEFGYSR

SEQ ID NO: 45 - DNA, 26 bp, Artificial Sequence
  Description of Artificial Sequence Synthetic primer
agatattgcacgggagaatatacaaa

SEQ ID NO: 46 - DNA, 27 bp, Artificial Sequence
  Description of Artificial Sequence Synthetic primer
tcaattcctgaaattaaagttcggata

SEQ ID NO: 47 - DNA, 23 bp, Artificial Sequence
  Description of Artificial Sequence Synthetic primer
tctgcagagttggaagcactcta

SEQ ID NO: 48 - DNA, 21 bp, Artificial Sequence
  Description of Artificial Sequence Synthetic primer
gccgaggcttttctaccagaa

SEQ ID NO: 49 - DNA, 20 bp, Artificial Sequence
  Description of Artificial Sequence Synthetic primer
catggcttgatcagcaagga

SEQ ID NO: 50 - DNA, 21 bp, Artificial Sequence
  Description of Artificial Sequence Synthetic primer
tggaagtgtgccctgaagaag

SEQ ID NO: 51 - DNA, 23 bp, Homo sapiens
caaggagctgacttcggaactaa

SEQ ID NO: 52 - DNA, 22 bp, Homo sapiens
agggaagacgatgtggttttca

SEQ ID NO: 53 - DNA, 22 bp, Homo sapiens
gggacatgtggagagcctactc

SEQ ID NO: 54 - DNA, 21 bp, Homo sapiens
catcatagttcccccgagcat

SEQ ID NO: 55 - DNA, 21 bp, Artificial Sequence
  Description of Artificial Sequence Synthetic primer
aagcagcaccagcaagtgaag

SEQ ID NO: 56 - DNA, 21 bp, Artificial Sequence
  Description of Artificial Sequence Synthetic primer
tcatggcctgtgtcagtcaaa

SEQ ID NO: 57 - DNA, 22 bp, Artificial Sequence
  Description of Artificial Sequence Synthetic primer
acatgccagccactgtgataga

SEQ ID NO: 58 - DNA, 21 bp, Artificial Sequence
  Description of Artificial Sequence Synthetic primer
ccctgccttcacaatgatctc

SEQ ID NO: 59 - DNA, 23 bp, Artificial Sequence
  Description of Artificial Sequence Synthetic primer
ggaattcacctcaagaacatcca

SEQ ID NO: 60 - DNA, 23 bp, Artificial Sequence
  Description of Artificial Sequence Synthetic primer
agtgtggctatgacttcggtttg

SEQ ID NO: 61 - DNA, 22 bp, Artificial Sequence
  Description of Artificial Sequence Synthetic primer
cagccacaagcagtccagatta

SEQ ID NO: 62 - DNA, 24 bp, Artificial Sequence
  Description of Artificial Sequence Synthetic primer
cctgactatcaatcacatcggaat

SEQ ID NO: 63 - DNA, 21 bp, Artificial Sequence
  Description of Artificial Sequence Synthetic primer
ccaggtgctccacatgacagt

SEQ ID NO: 64 - DNA, 24 bp, Artificial Sequence
  Description of Artificial Sequence Synthetic primer
aaacaaccaacaacaaggagaatg

SEQ ID NO: 65 - DNA, 21 bp, Artificial Sequence
  Description of Artificial Sequence Synthetic primer
cgtctccacacatcagcacaa

SEQ ID NO: 66 - DNA, 22 bp, Artificial Sequence
  Description of Artificial Sequence Synthetic primer
tcttggcagcaggatagtcctt

SEQ ID NO: 67 - DNA, 22 bp, Artificial Sequence
  Description of Artificial Sequence Synthetic primer
gcagaccagcatgacagatttc

SEQ ID NO: 68 - DNA, 20 bp, Artificial Sequence
  Description of Artificial Sequence Synthetic primer
gcggattagggcttcctctt

SEQ ID NO: 69 - DNA, 21 bp, Artificial Sequence
  Description of Artificial Sequence Synthetic primer
ggcaccagaggcagtaaccat

SEQ ID NO: 70 - DNA, 23 bp, Artificial Sequence
  Description of Artificial Sequence Synthetic primer
agcctctctggttctttcaatcg

SEQ ID NO: 71 - DNA, 19 bp, Artificial Sequence
  Description of Artificial Sequence Synthetic primer
tggttcacatcccgcggct

SEQ ID NO: 72 - DNA, 20 bp, Artificial Sequence
  Description of Artificial Sequence Synthetic primer
tggctcctcagtagcatcag

SEQ ID NO: 73 - DNA, 23 bp, Artificial Sequence
  Description of Artificial Sequence Synthetic primer
tgaagttcaatgcactggaactg

SEQ ID NO: 74 - DNA, 20 bp, Artificial Sequence
  Description of Artificial Sequence Synthetic primer
caggacgatctccacagcaa

SEQ ID NO: 75 - DNA, 23 bp, Artificial Sequence
  Description of Artificial Sequence Synthetic primer
tggagtccacgagatcatttaca

SEQ ID NO: 76 - DNA, 19 bp, Artificial Sequence
  Description of Artificial Sequence Synthetic primer
agccttggccctcggatat

SEQ ID NO: 77 - DNA, 21 bp, Artificial Sequence
  Description of Artificial Sequence Synthetic primer
cactgagttcgccaagagcat

SEQ ID NO: 78 - DNA, 23 bp, Artificial Sequence
  Description of Artificial Sequence Synthetic primer
cacgccatacttgagaagggtaa

SEQ ID NO: 79 - DNA, 23 bp, Artificial Sequence
  Description of Artificial Sequence Synthetic primer
gctagtgatcaacagtggcaatg

SEQ ID NO: 80 - DNA, 18 bp, Artificial Sequence
  Description of Artificial Sequence Synthetic primer
gctggcctctccgttgag

SEQ ID NO: 81 - DNA, 22 bp, Artificial Sequence
  Description of Artificial Sequence Synthetic primer
tgttcggtgtccagttccaata

SEQ ID NO: 82 - DNA, 22 bp, Artificial Sequence
  Description of Artificial Sequence Synthetic primer
tgccagtggtagagatggttga

SEQ ID NO: 83 - DNA, 22 bp, Artificial Sequence
  Description of Artificial Sequence Synthetic primer
acaactccaggaaggaaaccaa

SEQ ID NO: 84 - DNA, 19 bp, Artificial Sequence
  Description of Artificial Sequence Synthetic primer
cgaggactcctgcgagatg

SEQ ID NO: 85 - DNA, 23 bp, Artificial Sequence
  Description of Artificial Sequence Synthetic primer
tgaagaggagtggaggagacttg

SEQ ID NO: 86 - DNA, 24 bp, Artificial Sequence
  Description of Artificial Sequence Synthetic primer
gaatatgtggttctggctcatgaa

SEQ ID NO: 87 - DNA, 22 bp, Artificial Sequence
  Description of Artificial Sequence Synthetic primer
gagaaggagcgatctgctagct

SEQ ID NO: 88 - DNA, 23 bp, Artificial Sequence
  Description of Artificial Sequence Synthetic primer
cacgtagaagtgcaggtcatcag